(19) United States
(12) Patent Application Publication
(10) Pub. No.: US 2020/0237904 A1
(43) Pub. Date: Jul. 30, 2020

(54) Methods and Compositions Useful for Treating Cancer

(71) Applicant: Immune Therapeutics Inc., Orlando, FL (US)

(72) Inventors: Noreen Griffin, Winter Park, FL (US); Fengping Shan, Shenyang (CN)

(21) Appl. No.: 16/481,078

(22) PCT Filed: Jan. 26, 2018
(86) PCT No.: PCT/IB2018/050494

§ 371 Date: Jul. 26, 2019

(51) Int. Cl. A61K 39/39; A61K 38/22; A61K 31/7088; A61K 31/715; A61K 35/74; A61K 38/08; A61K 31/616; A61K 31/192; A61P 35/00; A61K 35/17; A61K 9/00

(52) U.S. Cl. CPC A61K 39/39; A61K 38/2292; A61K 31/7088; A61K 31/715; A61K 35/74; A61K 2039/55516; A61K 31/616; A61K 31/192; A61P 35/00; A61K 35/17; A61K 9/0019; A61K 38/08

(57) ABSTRACT

A cancer immunotherapy method for treating cancer in a patient comprising the steps of: removing lymphocytes from the peripheral blood of the patient; exposing the lymphocytes to one or more of i) a thymosin peptide, ii) a purified immune RNA or DNA. iii) one or more astragalus polysaccharides, iv) one or more letinous edodes polysaccharides, v) a cell wall component of Mycobacterium grannis, Mycobacterium phlei or Mycobaterium subtilis vi) a polysaccharide nucleic acid fraction of bacillus Calmette-Guerin, vii) a transfer factor viii) one or more non steroidal anti inflammatory agents and xi) an enkephalin chosen from methionine enkephalin or leucine enkephalin in vitro and infusing the exposed lymphocytes back into the patient is provided.

### FIELD OF THE INVENTION

The present invention relates to the treatment of mammals suffering from cancer by administering to a patient in need thereof: i) a thymosin peptide, ii) a purified immune RNA or DNA. iii) one or more astragalus polysaccharides, iv) one or more letinous edodes polysaccharides, v) a cell wall component of Mycobacterium grannis, Mycobacterium phlei or Mycobaterium subtilis vi) a polysaccharide nucleic acid fraction of bacillus Calmette-Guerin vii) a transfer factor and viii) one or more non steroidal anti inflammatory agents xi) an enkephalin chosen from methionine enkephalin or leucine enkephalin.

### BACKGROUND

Cancer continues to be a leading cause of mortality around the globe. Traditional regimens of cancer management have been successful in the management of a selective group of circulating and slow-growing solid cancers. However, many solid tumors are resistant to traditional approaches, and the prognosis in such cases is correspondingly poor.

One example is brain cancer. Each year, approximately 15,000 cases of high grade astrocytomas are diagnosed in the United States. The number is growing in both pediatric and adult populations. Standard treatments include cytoreductive surgery followed by radiation therapy or chemotherapy. There is no cure, and virtually all patients ultimately succumb to recurrent or progressive disease. The overall survival for grade IV astrocytomas (glioblastoma multiforme) is poor, with ˜50% of patients dying in the first year after diagnosis. Because these tumors are aggressive and highly resistant to standard treatments, new therapies are needed.

Another example is pancreatic cancer, the fifth leading cause of cancer-related deaths in the United States. The disease is associated with a high mortality rate, with a medium survival for untreated patients after diagnosis of about 4 months. Ninety percent of pancreatic cancer patients initially present with locally advanced, surgically un-resectable disease. Current therapy for these patients is strictly palliative and does not significantly impact on overall patient survival.

Another example is primary liver cancer which is one of the most common types of cancer in the world, with several hundred thousand new cases diagnosed annually. Of these, approximately 80% to 90% are hepatocellular carcinoma (HCC), also known as hepatoma. HCC is most prevalent in Asia and sub-Saharan Africa; in China alone there are more than 450,000 new cases per year, representing the fourth leading cause of death overall. HCC is the fastest increasing type of cancer in the United States: approximately 19,000 new cases are diagnosed annually, with resulting mortality of more than 16,000 per year.

Liver cancer commonly develops from liver cirrhosis, particularly that caused by hepatitis B or hepatitis C. The high incidence of liver cirrhosis and liver cancer found in Asia and Africa is due in large part to the high prevalence of hepatitis B and, increasingly, hepatitis C throughout these regions. The rising incidence of hepatitis C throughout the world is leading to an increase of liver cancer in other regions as well.

Cirrhosis due to causes other than hepatitis, including ingestion of ethanol and exposure to other organic solvents, can also lead to liver cancer. In addition, the ingestion of certain aflatoxins, which are toxins than can occur in stored grains and other foods as the result of fungal growth, is also associated with the development of liver cancer, as is the ingestion of other food- and water-borne carcinogens, including many drugs. Liver cancer may also arise from exposure to radiation, from mechanical trauma, and from other causes.

The current treatment of liver cancer is primarily surgical. If the cancer is localized to a particular region of the liver, and if that region can be safely removed, then surgical treatment may be successful. For the great majority of liver cancer patients, however, by the time the liver cancer is detected, too much of the liver is affected for surgical treatment to be safe and effective, and the cancer has commonly metastasized beyond the liver. Currently, these patients have few, if any, treatment options.

An emerging area of cancer treatment is immunotherapy. There are a number of immunological strategies under development, including: 1. Adoptive immunotherapy using stimulated autologous cells of various kinds; 2. Systemic transfer of allogeneic lymphocytes; 3. Vaccination at a distant site to generate a systemic tumor-specific immune response; 4.

Implantation of immune cells directly into the tumor.

The first of these strategies, adoptive immunotherapy, is directed towards providing the patient with a level of enhanced immunity by stimulating cells ex vivo, and then re-administering them to the patient. The cells are histo-compatible with the subject, and are generally obtained from a previous autologous donation.

One version is to stimulate autologous lymphocytes ex vivo with tumor-associated antigen to make them tumor-specific. Zarling et al. (1978) Nature 274:269-71 generated cytotoxic lymphocytes in vitro against autologous human leukemia cells. In U.S. Pat. No. 5,192,537, Osband suggests activating a tumor patient's mononuclear cells by culturing them ex vivo in the presence. of tumor cell extract and a non-specific activator like phytohemagglutinin or IL-1, and then treating the culture to deplete suppresser cell activity. Despite these experimental observations, systemic administration of ex vivo-stimulated autologous tumor-specific lymphocytes has not become part of standard cancer therapy.

Autologous lymphocytes and killer cells may also be stimulated non-specifically. In one example, Fc receptor expressing leukocytes that can mediate an antibody-dependent cell-mediated cytotoxicity reaction are generated by culturing with a combination of IL-2 and IFN-.gamma. (U.S. Pat. No. 5,308,626). In another example, peripheral blood-derived lymphocytes cultured in IL-2 form lymphokine-activated killer (LAK) cells, which are cytolytic towards a wide range of neoplastic cells, but not normal cells. In combination with high dose IL-2, LAK cells have had some success in the treatment of metastatic human melanoma and renal cell carcinoma. Rosenberg (1987) New Engl. J. Med. 316:889-897. For examples of trials conducted using LAK in the treatment of brain tumors, see Merchant et al. (1988) Cancer 62:665-671 & (1990) J. Neuro. Oncol. 8:173-198. While not associated with serious clinical complications, efficacy is typically only anecdotal or transient.

Another form of adoptive therapy using autologous cells has been proposed based on observations with tumor-infiltrating lymphocytes (TIL). TILs are obtained by collecting lymphocyte populations infiltrating into tumors, and culturing them ex vivo with IL-2. TILs have activity and tumor specificity superior to LAK cells, and have been experimentally administered, for example, to humans with advanced melanoma. Rosenberg et al. (1990) New Engl. J. Med. 323:570-578. Unfortunately, TILs can only be prepared in sufficient quantity to be clinically relevant in a limited number of tumor types, and remain experimental.

The second of the strategies for cancer immunotherapy listed earlier is adoptive transfer of allogeneic lymphocytes. The rationale of this experimental strategy is to create a general level of immune stimulation, and thereby overcome the anergy that prevents the host's immune system from rejecting the tumor. Strausser et al. (1981) J. Immunol. Vol. 127, No. 1 describe the lysis of human solid tumors by autologous cells sensitized in vitro to alloantigens. Zarling et al. (1978) Nature 274:269-71 demonstrated human anti-lymphoma responses in vivo following sensitization with allogeneic leukocytes. Kondo et al. (1984) Med Hypotheses 15:241-77 observed objective responses of this strategy in 20-30% of patients, and attributed the effect to depletion of suppressor T cells. The studies were performed on patients with disseminated or circulating disease. Even though these initial experiments were conducted over a decade ago, the strategy has not gained general acceptance, especially for the treatment of solid tumors.

The third of the immunotherapy strategies listed earlier is the generation of an active systemic tumor-speck immune response of host origin by administering a vaccine composition at a site distant from the tumor.

Various types of vaccines have been proposed, including isolated tumor-antigen vaccines and anti-idiotype vaccines. Another approach is to use tumor cells from the subject to be treated, or a derivative of such cells. For review see, Schirrmacher et al. (1995) J. Cancer Res. Clin. Oncol. 121:487-489. In U.S. Pat. No. 5,484,596, Hanna Jr. et al, claim a method for treating a resectable carcinoma to prevent recurrence or metastases, comprising surgically removing the tumor, dispersing the cells with collagenase, irradiating the cells, and vaccinating the patient with at least three consecutive doses of about 10⁷ cells.

In yet another approach, autologous or syngeneic tumor cells are genetically altered to produce a costimulatory molecule. For reviews see, Pardoll-et al. (1992) Curr. Opin. Immunol. 4:619-23; Salto et al. (1994) Cancer Res. 54:3516-3520; Vieweg et al. (1994) Cancer Res. 54:1760-1765; Gastl et al. (1992) Cancer Res. 52:6229-6236; and WO 96/07433. Tumor cells have been genetically altered to produce TNF-.alpha., IL-1, IL-2, IL-3, IL-4, IL-6, IL-7, IL-10, IFN-.alpha., IFN-.gamma. and GM-CSF.

PCT Publication No. WO 98/16238 describes cancer immunotherapy using autologous tumor cells combined with allogeneic cytokine-secreting cells. The vaccines comprise a source of tumor-associated antigen, particularly tumor cells from the patient to be treated, combined with an allogeneic cytokine-secreting cell line. Exemplary cytokines are IL-4, GM-CSF, IL-2, TNF-.alpha., and M-CSF in the secreted or membrane-bound form. The cytokine-producing cells provide immunostimulation in trans to generate a specific immune response against the tumor antigen. Vaccines can be tailored for each type of cancer or for each subject by mixing tumor antigen with an appropriate number of cytokine-producing cells, or with a cocktail of such cells producing a plurality of cytokines at a favorable ratio.

The fourth of the immunotherapy strategies listed earlier is intra-tumor implantation, directed at delivering effector cells directly to the site of action. The proximity of the effector cells to the target is supposed to promote the ability of the transplanted cells to react with the tumor, generating a graft versus tumor response.

Kruse et al. (Proc. Natl. Sci. USA, 87:9377-9381, 1990) analyzed various effector cell populations in adoptive immunotherapy of the 9L rat gliosarcoma cell line. Different cell populations were prepared that were designed to have a direct effector function—against the cancer cells. Included were syngeneic lymphocytes, nonadherent lymphocyte-activated killer (LAK) cells, adherent LAK cells, syngeneic cytotoxic T lymphocytes (CTL) raised against tumor antigens, and allogeneic CTL raised against alloantigens. The allogeneic cytotoxic T lymphocytes were claimed to prevent tumor take. The CTL were prepared by co-culturing thoracic duct lymphocytes from one inbred rat strain with spleen cells from rats syngeneic to the challenged animals, under conditions and for a period designed to enrich for cytotoxic effector cells. Treatment was effected by coinjecting the CTL with the tumor cells into the brains of rats in conjunction with recombinant IL-2, and then re-administering the CTL on two subsequent occasions. The regimen was claimed to forestall tumor take by 17 days. The authors state that the tumor is successful in the brain, because the brain is an immunologically privileged site which prevents the administered cells from being eliminated before they perform their function. A corollary of this is that the treatment would not be effective at other sites (such as the pancreas and the breast) that are not immunologically privileged.

In a subsequent study, Kruse et al. (J. Neuro-Oncol, 19:161-168, 1994) performed intracranial administrations of single or multiple source allogeneic cytotoxic T lymphocytes.

In this study, the 9L cancer cell line was injected into rats only 6 days before the initiation of treatment. A series of four injections of allogeneic T lymphocytes within the next 17 days was performed, and had the effect of extending the median life span of the rats by 19 days (about the same interval as the treatment protocol). There is no evidence for any lasting effect, despite the fact that four doses of the effector cells are given. This is consistent with the author's hypothesis that the tumoricidal effect is generated by the CTL themselves, and disappears once the administered cells are eliminated.

Two other publications by the same group demonstrates the natural progression of this CTL implantation technology in a direction towards greater enrichment for cells with a direct effector action against the tumor.

J. M. Redd, et al. Cancer Immunol. Immunother., 34:349, 1992 describe a method of generating allogeneic tumor-specific cytotoxic T lymphocytes. CTL were generated in culture from an inbred rat strain allogeneic to the tumor cell line. The cells were found to lyse both tumor cells and Con A stimulated lymphoblasts of the same tissue type. The tumor-specific subset was deliberately selected and enriched as being specific for a determinant expressed only by the tumor. The article concludes by stating that the ultimate goal of the authors is to transfer the technology to humans using allogeneic CTL lacking specificity for normal brain antigens (i.e., depleted of alloreactive cells). This is a significant elucidation of the previous article by Kruse et al. in Proc. Natl. Acad. Sci. (supra, p. 9579 col. 1), in which they refer to two types of allogeneic CTL, one of which is tumor specific and one of which is allospecific. The yield of tumor specific cells was substantially lower. The article by Redd et al. teaches that the tumor specific cells are preferred, and provides a way of enriching for them when using cultured rat cells.

More recently, Kruse et al. (Proc. Am. Assoc. Cancer Res. 36:474, 1995; FASEB J. 10:A1413, 1996) briefly outline a clinical study of human brain cancer patients. The patient's lymphocytes were expanded with OKT3 and IL-2, then co-cultured with allogeneic donor cells for 18-21 days in the presence of IL-2. Such culture conditions would result in a population highly enriched for terminally differentiated effector cells. Patients enrolled in the Phase I study received CTL into the tumor bed and were placed with a catheter for subsequent infusions. Ongoing treatment involved 1 to 5 treatment cycles every other month, with each cycle consisting of 2-3 CTL infusates within a 1 to 2 week period. Again, the ongoing necessity to re-administer the cells is consistent with the author's stated objective of providing cells with a direct cytolytic effect on the tumor.

The necessity of ongoing repeated administration of the effector cells to the tumor through a cannula severely limits the practical utility of this technology, both in terms of expense and the inconvenience to the patient.

In view of the limitations of may of these strategies, new approaches to the treatment of cancer are needed.

Considerable progress was made towards a simpler and more effective immunotherapeutic strategy by the development of cytoimplants. See PCT Publication No. WO 96/29394, a “Method for Treating Tumors”. Potent cellular compositions are placed directly into the tumor bed, leading to beneficial effects for patients with different types of cancers. The method can be conducted as follows: The tumor patient's leukocytes are co-cultured in a mixed lymphocyte cell reaction with healthy lymphocytes derived from an allogeneic donor. The alloactivated cells are surgically implanted at the tumor site, and produce a mixture of cytokines which induce a primary immune response. During this reaction, the host lymphoid cells identify both the graft lymphoid cells and tumor tissue as foreign.

### SUMMARY OF THE INVENTION

The present invention provides a composition comprising: i) a thymosin peptide, ii) a purified immune RNA or DNA. iii) one or more astragalus polysaccharides, iv) one or more letinous edodes polysaccharides, v) a cell wall component of Mycobacterium grannis, Mycobacterium phlei or Mycobaterium subtilis vi) a polysaccharide nucleic acid fraction of bacillus Calmette-Guerin vii) a transfer factor viii) one or more non steroidal anti inflammatory agents and ix) an enkephalin chosen from methionine enkephalin or leucine enkephalin.

Preferably the non steroidal anti inflammatory agent is chosen from the group consisting of aceclofenac, acemetacin, amoxiprin, aspirin, azapropazone, benorilate, bromfenac, carprofen, celecoxib, choline magnesium salicylate, diclofenac, diflunisal, etodolac, etoracoxib, faislamine, fenbuten, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinprazone, suprofen, tenoxicam, tiaprofenic acid, and tolmetin.

Preferably the non steroidal anti inflammatory agent is aspirin or ibuprofen.

Preferably the thymosin peptide is thymosin alpha 1 (TA1).

Preferably the enkephalin is methionine enkephalin.

Preferably the cell wall component is from Mycobacterium subtilis.

Preferably the composition, administration and dosing regime of the invention comprises:

Preferably the thymosin peptide is administered in an amount of about 1 and about 20 mg daily, preferably in an amount of about 2 to 10 mg daily, preferably in an amount of 4 mg. Preferably the thymosin peptide is administered IV or orally, preferably the thymosin peptide is administered for a period of up to two months.

It is further preferred that the thymosin peptide is administered by IV or orally, between about 1 and about 20 mg, preferably about 4 mg daily for about two months.

Preferably the purified immune RNA or DNA is administered in an amount of about 1 and about 20 mg daily, preferably in an amount of about 2 to 10 mg daily, preferably in an amount of 5 mg daily. Preferably the purified immune RNA or DNA is administered IV or orally, preferably the immune RNA or DNA is administered for a period of up to two months.

It is further preferred that immune RNA is administered by IV between about and about 20 mg preferably about 5 mg daily for about two months;

Preferably the astragalus polysaccharides are administered in an amount of about 1 and about 50 mg daily, preferably in an amount of about 2 to 30 mg daily, preferably in an amount of 20 mg daily. Preferably the astragalus polysaccharides are administered IV or orally, preferably the astragalus polysaccharides are administered for a period of up to two months.

It is further preferred that the astragalus polysaccharide is administered by IV or orally, between about 1 mg and about 50 mg preferably about 20 mg daily for about two months.

Preferably the letinous edodes polysaccharides are administered in an amount of about 1 and about 20 mg daily, preferably in an amount of about 2 to 15 mg daily, preferably in an amount of 10 mg daily. Preferably the letinous edodes polysaccharides are administered IV or orally, preferably the letinous edodes polysaccharides are administered for a period of up to two months.

It is further preferred that the letinous edodes polysaccharide is administered by IV or orally, between about 1 mg and about 20 mg preferably about 1.0 mg daily for about two months.

Preferably, the cell wall component of Mycobacterium grannis, Mycobacterium phlei or Mycobacterium subtilis, preferably utilins, is administered in an amount of about 0.5 and about 5 mg once a week, preferably in an amount of 1 mg once a week. Preferably, the cell wall component is administered IV or orally, preferably the cell wall component is administered for a period of up to two months.

It is further preferred that utilins are administered by IV, between about 0.5 mg and about 5 mg preferably about 1 mg once a week for two months.

Preferably the polysaccharide nucleic acid fraction of bacillus Calmette-Guerin (BCG) is administered in an amount of about 0.1 and about 10 mg in total, preferably in an amount of about 1 to 5 mg in total, preferably in an amount of 2 mg in total. Preferably the polysaccharide nucleic acid fraction of BCG is administered IV or orally, preferably the polysaccharide nucleic acid fraction of BCG is administered for a period of up to two months, more preferably the BCG is administered in an amount of 2 mg over three times in total, preferably on days 1, 3, and 5.

It is further preferred that the polysaccharide of BCG. administered by IV, between about 0.1 mg and about 10 mg preferably about 2 mg three times totally, on days 1, 3, and 5.

Preferably the transfer factor is administered in an amount of about 1 and about 20 mg daily, preferably in an amount of about 2 to 15 mg daily, preferably in an amount of 10 mg daily. Preferably the transfer factor is administered IV or orally, preferably the transfer factor is administered for a period of up to two months.

It is further preferred that the transfer factor administered orally, between about 1 mg and about 20 mg preferably about 10 mg daily for two months.

Preferably the one or more-nonsteroidal anti-inflammatory agents, preferably ibuprofen, are administered in an amount of about 100 and about 500 mg daily, preferably in an amount of about 200 to 400 mg daily, preferably in an amount of 300 mg daily. Preferably the one or more-nonsteroidal anti-inflammatory agents are administered IV or orally, preferably the one or more-nonsteroidal anti-inflammatory agents are administered for a period of up to 2 months, more preferred up to 5 days.

It is further preferred that ibuprofen is taken orally, between about 100 mg and 500 mg preferably about 300 mg daily for about 5 days.

Preferably the enkephalin chosen form methonine enkephalin or leucine enkephalin is administered in an amount of about 0.01 and about 100 mg daily, preferably in an amount of about 1 to 10 mg daily, preferably in an amount of 3 mg daily. Preferably the enkephalin is administered IV or orally, preferably the enkephalin is administered for a period of up to 2 months, more preferably up to 3 months.

It is further preferred that methionine encephalin is administered by IV between about 0.01 mg and about 100 mg preferably between about 1 mg and about 10 mg more preferably about 3 mg daily for about 2 months to 3 months.

The invention further provided a cancer immunotherapy method for treating cancer in a patient comprising the steps of:

a) separating lymphocytes from the peripheral blood of the patient;
b) exposing the lymphocytes to one or more of i) a thymosin peptide, ii) a purified immune RNA or DNA. iii) one or more astragalus polysaccharides, iv) one or more letinous edodes polysaccharides, v) a cell wall component of Mycobacterium grannis, Mycobacterium phlei or Mycobaterium subtilis vi) a polysaccharide nucleic acid fraction of bacillus Calmette-Guerin vii) a transfer factor viii) one or more non steroidal anti inflammatory agents and ix) an enkephalin chosen from methionine enkephalin or leucine enkephalin in vitro;
c) infusing the exposed lymphocytes back into the patient.

Preferably the removal step is performed by leukapheresis.

Preferably the cancer is chosen from the group consisting of malignant tumors, benign tumors, solid tumors, sarcomas, carcinomas, hyperproliferative disorders, carcinoids, Ewing sarcomas, Kaposi sarcomas, brain tumors, tumors originating from the brain and/or the nervous system and/or the meninges, gliomas, glioblastomas, neuroblastomas, stomach cancer, kidney cancer, kidney cell carcinomas, prostate cancer, prostate carcinomas, connective tissue tumors, soft tissue sarcomas, pancreas tumors, liver tumors, head tumors, neck tumors, laryngeal cancer, esophageal cancer, thyroid cancer, osteosarcomas, retinoblastomas, thymoma, testicular cancer, lung cancer, lung adenocarcinoma, small cell lung carcinoma, bronchial carcinomas, breast cancer, intestinal cancer, colorectal tumors, colon carcinomas, rectum carcinomas, gynaecological tumors, ovary tumors/ovarian tumors, uterine cancer, cervical cancer, cervix carcinomas, cancer of body of uterus, corpus carcinomas, endometrial carcinomas, urinary bladder cancer, urogenital tract cancer, bladder cancer, skin cancer, epithelial tumors, squamous epithelial carcinoma, basaliomas, spinaliomas, melanomas, intraocular melanomas, leukemias, monocyte leukemia, chronic leukemias, chronic myelotic leukemia, chronic lymphatic leukemia, acute leukemias, acute myelotic leukemia, acute lymphatic leukemia and lymphomas.

Preferably the cancer is liver cancer, stomach cancer and cervix carcinomas.

The invention further provides a method of treating cancer comprising administering to a patient i) a thymosin peptide, ii) a purified immune RNA or DNA. iii) one or more astragalus polysaccharides, iv) one or more letinous edodes polysaccharides, v) a cell wall component of Mycobacterium grannis, Mycobacterium phlei or Mycobaterium subtilis vi) a polysaccharide nucleic acid fraction of bacillus Calmette-Guerin vii) a transfer factor viii) one or more non steroidal anti inflammatory agents and ix) an enkephalin chosen from methionine enkephalin or leucine enkephalin.

Preferably the non steroidal anti inflammatory agent is chosen from the group consisting of aceclofenac, acemetacin, amoxiprin, aspirin, azapropazone, benorilate, bromfenac, carprofen, celecoxib, choline magnesium salicylate, diclofenac, diflunisal, etodolac, etoracoxib, faislamine, fenbuten, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinprazone, suprofen, tenoxicam, tiaprofenic acid, and tolmetin.

Preferably the non steroidal anti inflammatory agent is aspirin or ibuprofen.

Preferably the thymosin peptide is thymosin alpha 1 (TA1).

Preferably the enkephalin is methionine enkephalin.

Preferably the cell wall component is from Mycobacterium subtilis.

Preferably the cancer is chosen from the group consisting of malignant tumors, benign tumors, solid tumors, sarcomas, carcinomas, hyperproliferative disorders, carcinoids, Ewing sarcomas, Kaposi sarcomas, brain tumors, tumors originating from the brain and/or the nervous system and/or the meninges, gliomas, glioblastomas, neuroblastomas, stomach cancer, kidney cancer, kidney cell carcinomas, prostate cancer, prostate carcinomas, connective tissue tumors, soft tissue sarcomas, pancreas tumors, liver tumors, head tumors, neck tumors, laryngeal cancer, esophageal cancer, thyroid cancer, osteosarcomas, retinoblastomas, thymoma, testicular cancer, lung cancer, lung adenocarcinoma, small cell lung carcinoma, bronchial carcinomas, breast cancer, intestinal cancer, colorectal tumors, colon carcinomas, rectum carcinomas, gynaecological tumors, ovary tumors/ovarian tumors, uterine cancer, cervical cancer, cervix carcinomas, cancer of body of uterus, corpus carcinomas, endometrial carcinomas, urinary bladder cancer, urogenital tract cancer, bladder cancer, skin cancer, epithelial tumors, squamous epithelial carcinoma, basaliomas, spinaliomas, melanomas, intraocular melanomas, leukemias, monocyte leukemia, chronic leukemias, chronic myelotic leukemia, chronic lymphatic leukemia, acute leukemias, acute myelotic leukemia, acute lymphatic leukemia and lymphomas.

Preferably the cancer is liver cancer, stomach cancer or cervix carcinomas.

Preferably the purified immune RNA or DNA is in injection form.

Preferably the cell wall component of Mycobacterium grannis, Mycobacterium phlei or Mycobaterium subtilis is in injection form.

Preferably the cell wall component is from Mycobaterium subtilis.

Preferably the polysaccharide nucleic acid fraction of bacillus Calmette-Guerin is in injection form.

Preferably the enkephalin chosen from methionine enkephalin or leucine enkephalin is in injection form.

Preferably the enkephalin is methionine enkephalin.

Preferably the thymosin peptide, one or more astragalus polysaccharides, one or more letinous edodes polysaccharides and the transfer factor are each in tablet form.

The invention further provides a method of treating cancer where the cancer is chosen from the group consisting of malignant tumors, benign tumors, solid tumors, sarcomas, carcinomas, hyperproliferative disorders, carcinoids, Ewing sarcomas, Kaposi sarcomas, brain tumors, tumors originating from the brain and/or the nervous system and/or the meninges, gliomas, glioblastomas, neuroblastomas, stomach cancer, kidney cancer, kidney cell carcinomas, prostate cancer, prostate carcinomas, connective tissue tumors, soft tissue sarcomas, pancreas tumors, liver tumors, head tumors, neck tumors, laryngeal cancer, esophageal cancer, thyroid cancer, osteosarcomas, retinoblastomas, thymoma, testicular cancer, lung cancer, lung adenocarcinoma, small cell lung carcinoma, bronchial carcinomas, breast cancer, intestinal cancer, colorectal tumors, colon carcinomas, rectum carcinomas, gynaecological tumors, ovary tumors/ovarian tumors, uterine cancer, cervical cancer, cervix carcinomas, cancer of body of uterus, corpus carcinomas, endometrial carcinomas, urinary bladder cancer, urogenital tract cancer, bladder cancer, skin cancer, epithelial tumors, squamous epithelial carcinoma, basaliomas, spinaliomas, melanomas, intraocular melanomas, leukemias, monocyte leukemia, chronic leukemias, chronic myelotic leukemia, chronic lymphatic leukemia, acute leukemias, acute myelotic leukemia, acute lymphatic leukemia and lymphomas.

The invention also provides that the mammal is a human.

The invention also provides that the administration route is chosen from the group consisting of oral, sublingual, subcutaneous, intramuscular, intravenous, topical, local, intratracheal, intranasal, transdermal and rectal administration.

The present invention provides i) a thymosin peptide, ii) a purified immune RNA or DNA. iii) one or more astragalus polysaccharides, iv) one or more letinous edodes polysaccharides, v) a cell wall component of Mycobacterium grannis, Mycobacterium phlei or Mycobaterium subtilis vi) a polysaccharide nucleic acid fraction of bacillus Calmette-Guerin vii) a transfer factor and viii) one or more non steroidal anti inflammatory agents xi) an enkephalin chosen from methionine enkephalin or leucine enkephalin for use as a medicament.

The present invention provides i) a thymosin peptide, ii) a purified immune RNA or DNA. iii) one or more astragalus polysaccharides, iv) one or more letinous edodes polysaccharides, v) a cell wall component of Mycobacterium grannis, Mycobacterium phlei or Mycobaterium subtilis vi) a polysaccharide nucleic acid fraction of bacillus Calmette-Guerin vii) a transfer factor and viii) one or more non steroidal anti inflammatory agents xi) an enkephalin chosen from methionine enkephalin or leucine enkephalin for use in the treatment of cancer.

### BRIEF DESCRIPTION OF THE DRAWINGS

FIG. 1 shows a tumor animal model with S-180 cells. After administration of an IV injection with the composition of the invention, once a day for 7 successive days, the tumor size was measured. FIG. 1a shows a mouse tumor prior to treatment and FIG. 1b after treatment with a composition of the invention showing tumor shrinkage.

FIG. 2 shows the tumor size of S-180 carcinoma removed from mice before treatment with a preferred composition of the invention (FIG. 2a), after treatment with a preferred composition of the invention (FIG. 2b) and after treatment with a normal saline solution

FIG. 3a-d show dendritic cells before (FIGS. 3a and c) and after treatment (3b and d) with the composition visualized by scanning electronic microscope (SEM) to determine the maturation degree.

FIG. 4 shows an MRI of patient 1 before administration of a preferred composition of the invention (FIG. 4a) and after administration (FIG. 4b), showing tumor shrinkage

FIG. 5 shows an MRI of patient 2 before administration of a preferred composition of the invention (FIG. 5a) and after administration (FIG. 5b).

FIG. 6 shows an MRI of patient 3 before administration of a preferred composition of the invention (FIGS. 6a and 6c) and after administration (FIGS. 6b and 6d).

FIG. 7 shows an MRI of patient 4 before administration of a preferred composition of the invention (FIG. 7a) and after administration (FIG. 7b).

FIG. 8 shows stained blood cells of patient 5 before administration of a preferred composition of the invention (FIG. 8a) and after administration (FIG. 8b).

### TERMS AND DEFINITIONS USED

The components of the formulation may be formulated for administration in any convenient way for use in human or veterinary medicine and the invention therefore includes within its scope pharmaceutical compositions comprising an ingredient of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

### Routes of Administration and Unit Dosage Forms

The routes for administration include oral (e.g., as a tablet, capsule, or as an ingestible solution), topical, mucosal (e.g., as a nasal spray or aerosol for inhalation), nasal, parenteral (e.g., by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual. The compositions of the invention may be especially formulated for any of those administration routes. In preferred embodiments, the pharmaceutical compositions of the invention are formulated in a form that is suitable for oral delivery.

There may be different composition/formulation requirements depending on the different delivery systems. It is to be understood that not all of the compounds need to be administered by the same route. Likewise, if the composition comprises more than one active component, then those components may be administered by different routes. By way of example, the pharmaceutical composition of the invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by multiple routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile. For example, the compound of Formula I may be coated with an enteric coating layer. The enteric coating layer material may be dispersed or dissolved in either water or in a suitable organic solvent. As enteric coating layer polymers, one or more, separately or in combination, of the following can be used; e.g., solutions or dispersions of methacrylic acid copolymers, cellulose acetate phthalate, cellulose acetate butyrate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s). For environmental reasons, an aqueous coating process may be preferred. In such aqueous processes methacrylic acid copolymers are most preferred.

When appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For buccal or sublingual administration, the compositions may be administered in the form of tablets or lozenges, which can be formulated in a conventional manner.

When the composition of the invention is to be administered parenterally, such administration includes one or more of: intravenously, intraarterially, intraperitoneally, intrathecally, intraventricularly, intraurethrally, intrasternally, intracranially, intramuscularly or subcutaneously administering the agent; and/or by using infusion techniques.

Pharmaceutical compositions of the invention can be administered parenterally, e.g., by infusion or injection. Pharmaceutical compositions suitable for injection or infusion may be in the form of a sterile aqueous solution, a dispersion or a sterile powder that contains the active ingredient, adjusted, if necessary, for preparation of such a sterile solution or dispersion suitable for infusion or injection. This preparation may optionally be encapsulated into liposomes. In all cases, the final preparation must be sterile, liquid, and stable under production and storage conditions. To improve storage stability, such preparations may also contain a preservative to prevent the growth of microorganisms. Prevention of the action of micro-organisms can be achieved by the addition of various antibacterial and antifungal agents, e.g., paraben, chlorobutanol, or acsorbic acid. In many cases isotonic substances are recommended, e.g., sugars, buffers and sodium chloride to assure osmotic pressure similar to those of body fluids, particularly blood. Prolonged absorption of such injectable mixtures can be achieved by introduction of absorption-delaying agents, such as aluminium monostearate or gelatin.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

For parenteral administration, the compound is best used in the form of a sterile aqueous solution, which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Sterile injectable solutions can be prepared by mixing a compound of formula I with an appropriate solvent and one or more of the aforementioned carriers, followed by sterile filtering. In the case of sterile powders suitable for use in the preparation of sterile injectable solutions, preferable preparation methods include drying in vacuum and lyophilization, which provide powdery mixtures of the aldosterone receptor antagonists and desired excipients for subsequent preparation of sterile solutions.

The compounds according to the invention may be formulated for use in human or veterinary medicine by injection (e.g., by intravenous bolus injection or infusion or via intramuscular, subcutaneous or intrathecal routes) and may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain formulatory agents such as suspending, stabilizing, solubilizing and/or dispersing agents. Alternatively, the active ingredient may be in sterile powder form for reconstitution with a suitable vehicle, e.g., sterile, pyrogen-free water, before use.

The composition can be administered (e.g., orally or topically) in the form of tablets, capsules, ovules, elixirs, solutions or suspensions, which may contain flavoring or coloring agents, for immediate-, delayed-, modified-, sustained-, pulsed- or controlled-release applications.

The composition may also be presented for human or veterinary use in a form suitable for oral or buccal administration, for example in the form of solutions, gels, syrups, mouth washes or suspensions, or a dry powder for constitution with water or other suitable vehicle before use, optionally with flavoring and coloring agents. Solid compositions such as tablets, capsules, lozenges, pastilles, pills, boluses, powder, pastes, granules, bullets or premix preparations may also be used. Solid and liquid compositions for oral use may be prepared according to methods well-known in the art. Such compositions may also contain one or more pharmaceutically acceptable carriers and excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

The compositions may be administered orally, in the form of rapid or controlled release tablets, microparticles, mini tablets, capsules, sachets, and oral solutions or suspensions, or powders for the preparation thereof. In addition to the new solid-state forms of pantoprazole of the invention as the active substance, oral preparations may optionally include various standard pharmaceutical carriers and excipients, such as binders, fillers, buffers, lubricants, glidants, dyes, disintegrants, odourants, sweeteners, surfactants, mold release agents, antiadhesive agents and coatings. Some excipients may have multiple roles in the compositions, e.g., act as both binders and disintegrants.

Examples of pharmaceutically acceptable disintegrants for oral compositions include starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and cross-linked polyvinylpyrrolidone.

Examples of pharmaceutically acceptable binders for oral compositions include acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulphate.

Examples of pharmaceutically acceptable lubricants useful in the compositions of the invention include magnesium stearate, talc, polyethylene glycol, polymers of ethylene oxide, sodium lauryl sulphate, magnesium lauryl sulphate, sodium oleate, sodium stearyl fumarate, and colloidal silicon dioxide.

Examples of suitable pharmaceutically acceptable odourants for the oral compositions include synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include hydroxypropylmethylcellulose, hydroxypropylcellulose and acrylate-methacrylate copolymers.

Examples of pharmaceutically acceptable sweeteners for the oral compositions include aspartame, saccharin, saccharin sodium, sodium cyclamate, xylitol, mannitol, sorbitol, lactose and sucrose.

Examples of pharmaceutically acceptable buffers include citric acid, sodium citrate, sodium bicarbonate, dibasic sodium phosphate, magnesium oxide, calcium carbonate and magnesium hydroxide.

Examples of pharmaceutically acceptable surfactants include sodium lauryl sulphate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

The composition may also, for example, be formulated as suppositories e.g., containing conventional suppository bases for use in human or veterinary medicine or as pessaries e.g., containing conventional pessary bases.

The composition may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g., eye ear or nose drops) or pour-ons.

For application topically to the skin, the agent of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax, sorbitan monostearate, a polyethylene glycol, liquid paraffin, polysorbate 60, cetyl esters wax, cetearyl alcohol, 2-octyldodecanol, benzyl alcohol, and water. Such compositions may also contain other pharmaceutically acceptable excipients, such as polymers, oils, liquid carriers, surfactants, buffers, preservatives, stabilizers, antioxidants, moisturizers, emollients, colourants, and odourants.

Examples of pharmaceutically acceptable polymers suitable for such topical compositions include acrylic polymers; cellulose derivatives, such as carboxymethylcellulose sodium, methylcellulose or hydroxypropylcellulose; natural polymers, such as alginates, tragacanth, pectin, xanthan and cytosan.

Examples of suitable pharmaceutically acceptable oils which are so useful include mineral oils, silicone oils, fatty acids, alcohols, and glycols.

Examples of suitable pharmaceutically acceptable liquid carriers include water, alcohols or glycols such as ethanol, isopropanol, propylene glycol, hexylene glycol, glycerol and polyethylene glycol, or mixtures thereof in which the pseudopolymorph is dissolved or dispersed, optionally with the addition of non-toxic anionic, cationic or non-ionic surfactants, and inorganic or organic buffers.

Examples of pharmaceutically acceptable preservatives include sodium benzoate, ascorbic acid, esters of p-hydroxybenzoic acid and various antibacterial and antifungal agents such as solvents, for example ethanol, propylene glycol, benzyl alcohol, chlorobutanol, quaternary ammonium salts, and parabens (such as methyl paraben, ethyl paraben and propyl paraben).

Examples of pharmaceutically acceptable stabilizers and antioxidants include ethylenediaminetetraacetic acid (EDTA), thiourea, tocopherol and butyl hydroxyanisole.

Examples of pharmaceutically acceptable moisturizers include glycerine, sorbitol, urea and polyethylene glycol.

Examples of pharmaceutically acceptable emollients include mineral oils, isopropyl myristate, and isopropyl palmitate.

The compounds may also be dermally or transdermally administered, for example, by use of a skin patch.

For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride.

The pharmaceutical composition or unit dosage form of the invention may be administered according to a dosage and administration regimen defined by routine testing in the light of the guidelines given above in order to obtain optimal activity while minimizing toxicity or side effects for a particular patient. However, such fine tuning of the therapeutic regimen is routine in the light of the guidelines given herein.

The dosage of the active agents of the invention may vary according to a variety of factors such as underlying disease conditions, the individual's condition, weight, gender and age, and the mode of administration. An effective amount for treating a disorder can easily be determined by empirical methods known to those of ordinary skill in the art, for example by establishing a matrix of dosages and frequencies of administration and comparing a group of experimental units or subjects at each point in the matrix. The exact amount to be administered to a patient will vary depending on the state and severity of the disorder and the physical condition of the patient. A measurable amelioration of any symptom or parameter can be determined by a person skilled in the art or reported by the patient to the physician.

The pharmaceutical composition or unit dosage form may be administered in a single daily dose, or the total daily dosage may be administered in divided doses. In addition, co-administration or sequential administration of another compound for the treatment of the disorder may be desirable. To this purpose, the combined active principles are formulated into a simple dosage unit.

For combination treatment where the compounds are in separate dosage formulations, the compounds can be administered concurrently, or each can be administered at staggered intervals. For example, the compound of the invention may be administered in the morning and the antimuscarinic compound may be administered in the evening, or vice versa. Additional compounds may be administered at specific intervals too. The order of administration will depend upon a variety of factors including age, weight, gender and medical condition of the patient; the severity and aetiology of the disorders to be treated, the route of administration, the renal and hepatic function of the patient, the treatment history of the patient, and the responsiveness of the patient. Determination of the order of administration may be fine-tuned and such fine-tuning is routine in the light of the guidelines given herein.

### DETAILED DESCRIPTION OF THE INVENTION

In accordance with the present invention, a cancer immunotherapy method for treating cancer in a patient comprising the steps of:
a) separating lymphocytes from the peripheral blood of the patient;
b) exposing the lymphocytes to one or more of i) a thymosin peptide, ii) a purified immune RNA or DNA. iii) one or more astragalus polysaccharides, iv) one or more letinous edodes polysaccharides, v) a cell wall component of Mycobacterium grannis, Mycobacterium phlei or Mycobaterium subtilis vi) a polysaccharide nucleic acid fraction of bacillus Calmette-Guerin, vii) a transfer factor viii) one or more non steroidal anti inflammatory agents and ix) an enkephalin chosen from methionine enkephalin or leucine enkephalin in vitro and:
c) infusing the exposed lymphocytes back into the patient is provided.

The invention also provides a method of treating cancer comprising administering to a patient i) a thymosin peptide, ii) a purified immune RNA or DNA. iii) one or more astragalus polysaccharides, iv) one or more letinous edodes polysaccharides, v) a cell wall component of Mycobacterium grannis, Mycobacterium phlei or Mycobaterium subtilis vi) a polysaccharide nucleic acid fraction of bacillus Calmette-Guerin vii) a transfer factor viii) one or more non steroidal anti inflammatory agents and ix) an enkephalin chosen from methionine enkephalin or leucine enkephalin.

Alternatively, cancer patients can be treated through direct administration of agents mentioned above. The preferred composition, dosing regime and amounts are as follows:

(1) thymosin, IV or orally, 4 mg daily for two months;
(2) purified immune RNA, IV, 5 mg daily for two months;
(3) astragalus polysaccharide, IV or orally, 20 mg daily for two months;
(4) transfer factor, orally, 10 mg daily for two months;
(5) cell wall components, e.g. utilins, IV, 1 mg once a week for two months;
(6) polysaccharide of BCG, IV, three times, 2 mg in total, on days 1, 3, and 5;
(7) letinous edodes polysaccharide, IV or orally, 10 mg daily for two months;
(8) methionine encephalin, IV, 3 mg daily for three months; and
(9) ibuprofen, orally, 300 mg daily for 5 days.

The following examples provide a detailed illustration of the method of the present invention. These examples are not intended to limit or restrict the scope of the invention in any way.

The components of the composition invention are available from numerous commercial sources.

### Examples

### 1. Impact on Lymphocytes Proliferation in Cancer Patients

50 ml peripheral blood from a cancer patient was loaded on the upper layer of a Ficoll lymphocyte separation solution in a centrifuge tube and centrifuged at 1.500 rpm for 10 min. The white lymphocyte layer was removed with a pipette and the white lymphocyte layer was transferred cells were transferred into a flask. To the cells, the composition of the invention was added to dilute the cell number to 10⁵ cells/ml. The cells were cultured in an incubator at 37° C. for 24 h, whereby RPMI 1640 medium was used as a control. Increments of lymphocytes subpopulations where determined with flow cytometry using a BD Biosciences flow cytometer.

### Average Percentage of Lymphocytes Subpopulations

After treating blood from 20 cancer patients with the composition of the invention, the following results were obtained using a BD Biosciences flow cytometer.

| | Control | Treatment | P |
|---|---|---|---|
| CD3+ T cell | 49.90 ± 4.06 | 66.04 ± 4.20 | P < 0.01 |
| CD8+ T cell | 33.33 ± 3.62 | 56.63 ± 3.78 | P < 0.01 |
| CD4+ T cell | 20.65 ± 3.48 | 33.81 ± 5.78 | P < 0.01 |
| NK cell | 7.98 ± 1.59 | 25.39 ± 4.42 | P < 0.01 |
| Treg cell | 4.62 ± 0.64 | 2.85 ± 0.48 | P < 0.01 |
| Total nucleated cell | 1.10 ± 0.13 | 4.01 ± 0.75 | P < 0.01 |

### 2. Testing Anticancer Activity of the Composition on Mice.

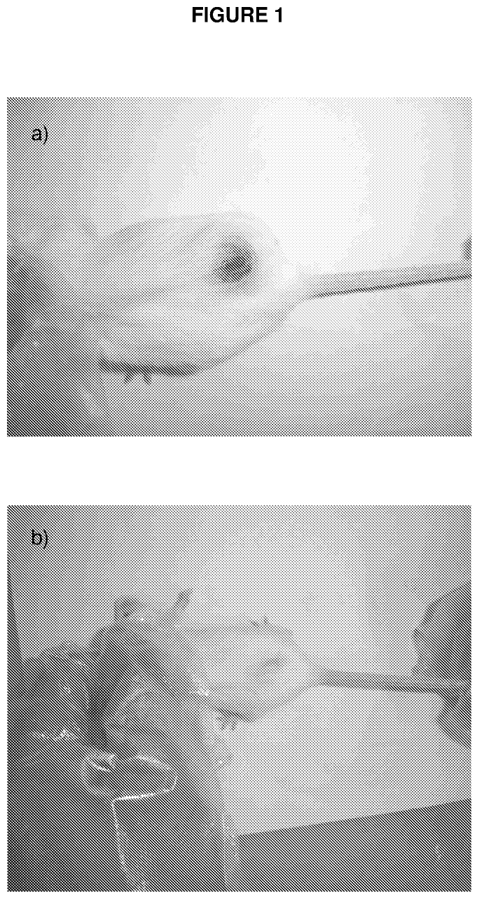

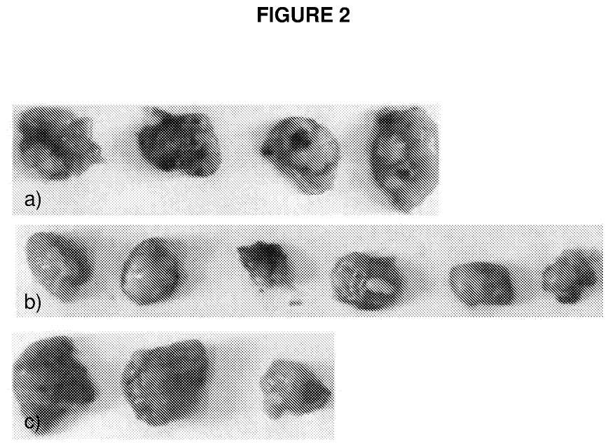

A tumor animal model was created with S-180 cells. An IV injection was administered with the preferred composition of the invention, once a day for 7 successive days and measure tumor size. Female C57BL/6 mice were obtained from Harlan SLAC Laboratory Animals Co. Ltd (Shanghai, China) (FIG. 1a). Mice were housed in the pathogen-free animal house at China Medical University and were used at 6-8 weeks of age. All experiments with animals were conducted in accordance with the Guide for the Care and Use of Laboratory Animals as adopted and promulgated by the China National Institutes of Health. Mice were inoculated with 10⁶ S180 cells subcutaneously to create a cancer model. After ending of treatment, the sizes of tumors were measured (FIGS. 1b and 2a-c).

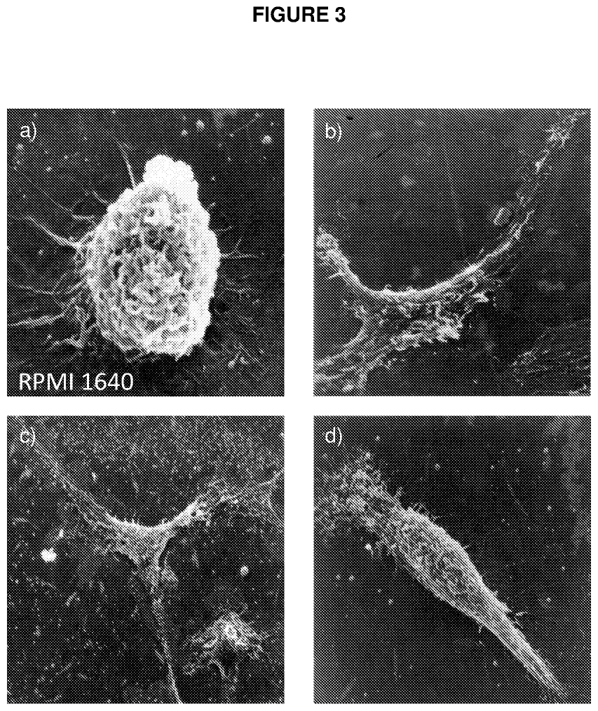

3. Dendritic Cells Before and after Treatment with the Composition Separated dendritic cells were taken after treatment with the preferred composition of the invention followed by centrifugation and re-suspension in 0.5 ml 0.05 M pH 7.2 phosphorus buffer solution. The cells were then fixed in 2.5% glutaraldehyde, by adding 1% osmium tetroxide and allowing to stand overnight, dehydrated in ethanol and embedded in EPON resin. Sections were made on a Reiehert-Jung Ultracut E, stained with uranyl acetate and lead citrate. The cells were then viewed using a scanning electron microscope to determine degree of maturation (FIGS. 3a-d).
4. Treatment of Cancer Patients with the Composition Using Leukapharesis

Regime: Before treatment all cancer patients underwent CT scans (GECT/e USA). Cancer patients were then administered with the invention through iv or orally, every day for one month, from second month on taking the preferred composition of the invention every other day for another one month:

(1) thymosin, IV or orally, 4 mg daily for two months;
(2) purified immune RNA, IV, 5 mg daily for two months;
(3) astragalus polysaccharide, IV or orally, 20 mg daily for two months;
(4) transfer factor, orally, 10 mg daily for two months;
(5) cell wall components, e.g. utilins, IV, 1 mg once a week for two months;
(6) polysaccharide of BCG, IV, three times, 2 m (correct???YES), on days 1, 3, and 5;
(7) letinous edodes polysaccharide, IV or orally, 10 mg daily for two months;
(8) methionine encephalin, IV, 3 mg daily for three months; and
(9) ibuprofen, orally, 300 mg daily for 5 days.

After ending of treatment for two months, a CT scan was taken again and at the same time lymphocytes sub-populations were measured with flow cytometry.

Patient 1: a 55 year old female, with terminal lung cancer, treated with a composition of the invention via leukopharesis (white cells separated from blood and exposed to a preferred composition of the invention and reintroduced to the patient) four times in two months and by administration of a preferred composition using the preferred dosing:

(1) thymosin, IV or orally, 4 mg daily for two months;
(2) purified immune RNA, IV, 5 mg daily for two months;
(3) astragalus polysaccharide, IV or orally, 20 mg daily for two months;
(4) transfer factor, orally, 10 mg daily for two months;
(5) cell wall components, e.g. utilins, IV, 1 mg once a week for two months;
(6) polysaccharide of BCG, IV, three times, 2 mg in total on days 1, 3, and 5;
(7) letinous edodes polysaccharide IV or ora mg daily for two months;
(8) methionine encephalin, IV, 3 mg daily for three months; and
(9) ibuprofen, orally, 300 mg daily for 5 days.

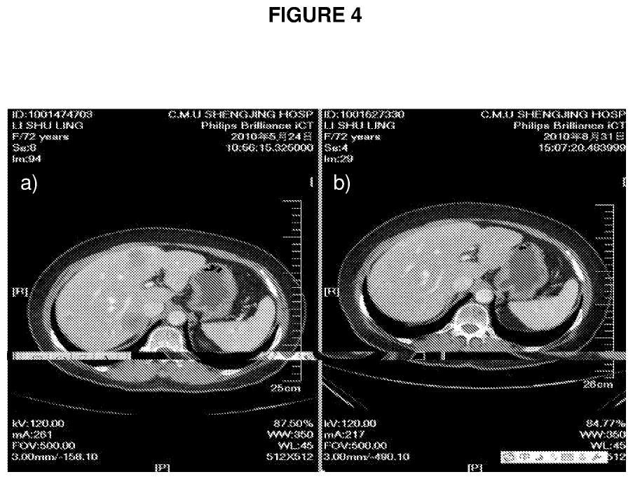

Tumor shrinkage was observed as evidenced by the MRI shown in FIG. 4.

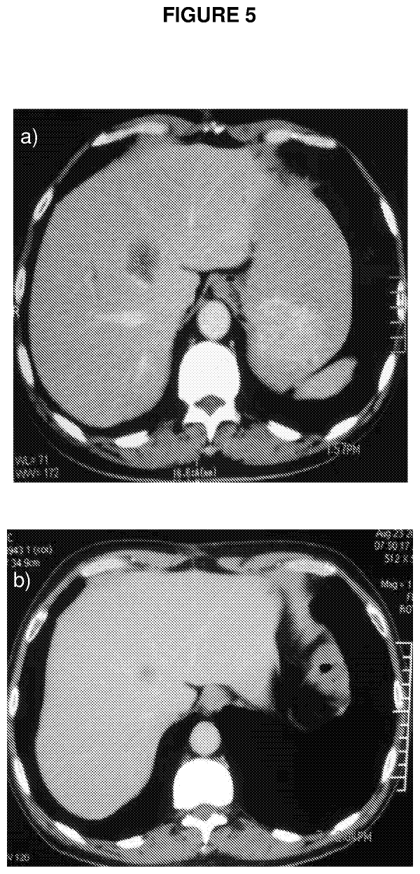

Patient a 56 year old male, with severe terminal liver cancer metastasized from rectal cancer treated with a composition of the invention leukopharesis (white cells separated from blood and exposed to a preferred composition of the invention and reintroduced to the patient) four times in two months and by administration of a preferred composition using the preferred dosing. Tumor shrinkage was observed as evidenced by the MRI in FIG. 5.

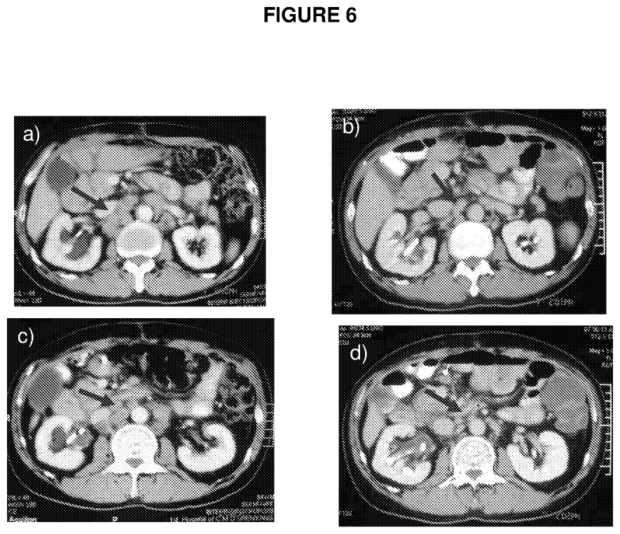

Patient 3: a 47 year old female with terminal lymphoma, treated with a composition of the invention via leukapharesis (white cells separated from blood and exposed to a preferred composition of the invention and reintroduced to the patient) four times in two months and by administration of a preferred composition using the preferred dosing. Tumor shrinkage observed as evidenced by the MRI shown in FIG. 6.

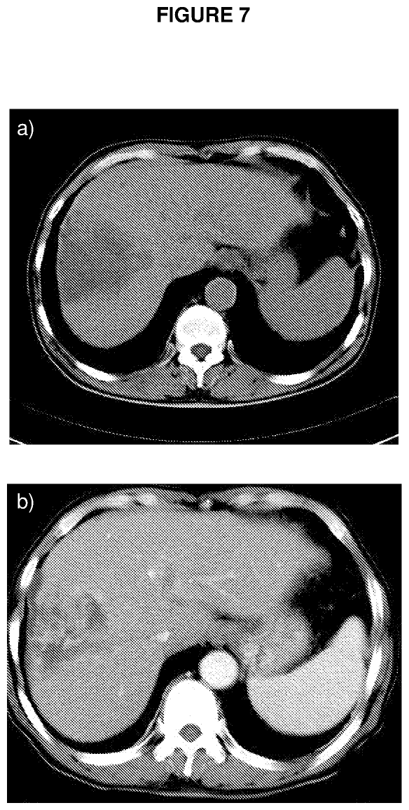

Patient 4: a 57 year old male with severe terminal liver cancer, treated with leukapharesis (white cells separated from blood and exposed to a preferred composition of the invention and reintroduced to the patient) using the composition of the invention four times in two months and by administration of a preferred composition using the preferred dosing. Tumor shrinkage was observed as evidenced by the MRI shown in FIG. 7.

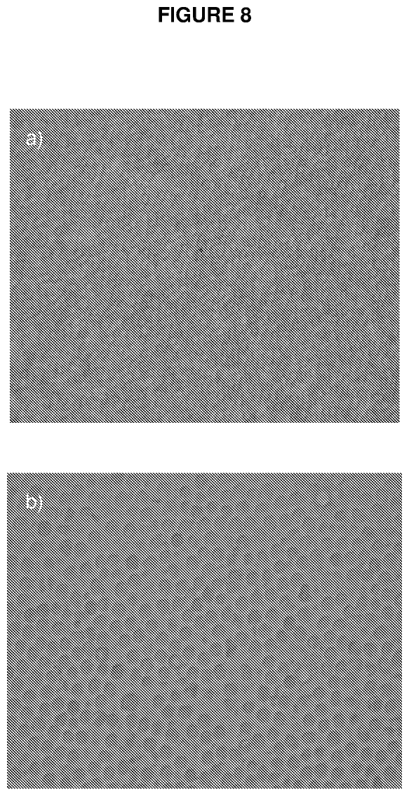

Patient 5 a 25 year old male with severe terminal leukemia was treated with leukapharesis (white cells separated from blood and exposed to a preferred composition of the invention and reintroduced to the patient) with the composition four times in two months and by administration of a preferred composition using the preferred dosing. Tumor cells returned to normal as evidenced by microscopic examination shown in FIG. 8.

Patient 6 a woman, 53, with terminal cervical cancer and metastasized to whole body and with ascites. Post treatment with agents above for three months the patient removed and all ascites disappeared. Immune system returned to normal.

Patient 7 a man 66, with terminal stomach sinus cancer and digestive track blockage caused by cancer. Patient was treated with a preferred composition of the for four months with tumor shrinkage observed.

1. A composition comprising: i) a thymosin peptide, ii) a purified immune RNA or DNA. iii) one or more astragalus polysaccharides, iv) one or more letinous edodes polysaccharides, v) a cell wall component of Mycobacterium grannis, Mycobacterium phlei or Mycobaterium subtilis vi) a polysaccharide nucleic acid fraction of bacillus Calmette-Guerin vii) a transfer factor viii) one or more non steroidal anti inflammatory agents and ix) an enkephalin chosen from methionine enkephalin or leucine enkephalin.

2. The composition of claim 1, wherein the thymosin peptide is thymosin alpha 1 (TA1).

3. The composition of claim 1, wherein the enkephalin is methionine enkephalin.

4. The composition of claim 1, wherein the cell wall component is from Mycobacterium subtilis.

5. The composition of claim 1, wherein the non steroidal anti inflammatory agent is chosen from the group consisting of aceclofenac, acemetacin, amoxiprin, aspirin, azapropazone, benorilate, bromfenac, carprofen, celecoxib, choline magnesium salicylate, diclofenac, diflunisal, etodolac, etoracoxib, faislamine, fenbuten, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinprazone, suprofen, tenoxicam, tiaprofenic acid, and tolmetin.

6. The composition according to claim 5, wherein the non steroidal anti inflammatory agent is chosen from asprin and ibuprofen.

7. A cancer immunotherapy method for treating cancer in a patient, comprising the steps of:
a) separating lymphocytes from the peripheral blood of the patient;
b) exposing the lymphocytes to one or more of i) a thymosin peptide, ii) a purified immune RNA or DNA. iii) one or more astragalus polysaccharides, iv) one or more letinous edodes polysaccharides, v) a cell wall component of Mycobacterium grannis, Mycobacterium phlei or Mycobaterium subtilis vi) a polysaccharide nucleic acid fraction of bacillus Calmette-Guerin, vii) a transfer factor viii) one or more non steroidal anti inflammatory agents and xi) an enkephalin chosen from methionine enkephalin or leucine enkephalin in vitro and:
c) infusing the exposed lymphocytes back into the patient.

8. The method of claim 7, wherein the removal step is performed by leukapheresis.

9. The method of claim 7, wherein the cancer is chosen from the group consisting of malignant tumors, benign tumors, solid tumors, sarcomas, carcinomas, hyperproliferative disorders, carcinoids, Ewing sarcomas, Kaposi sarcomas, brain tumors, tumors originating from the brain and/or the nervous system and/or the meninges, gliomas, glioblastomas, neuroblastomas, stomach cancer, kidney cancer, kidney cell carcinomas, prostate cancer, prostate carcinomas, connective tissue tumors, soft tissue sarcomas, pancreas tumors, liver tumors, head tumors, neck tumors, laryngeal cancer, esophageal cancer, thyroid cancer, osteosarcomas, retinoblastomas, thymoma, testicular cancer, lung cancer, lung adenocarcinoma, small cell lung carcinoma, bronchial carcinomas, breast cancer, intestinal cancer, colorectal tumors, colon carcinomas, rectum carcinomas, gynaecological tumors, ovary tumors/ovarian tumors, uterine cancer, cervical cancer, cervix carcinomas, cancer of body of uterus, corpus carcinomas, endometrial carcinomas, urinary bladder cancer, urogenital tract cancer, bladder cancer, skin cancer, epithelial tumors, squamous epithelial carcinoma, basaliomas, spinaliomas, melanomas, intraocular melanomas, leukemias, monocyte leukemia, chronic leukemias, chronic myelotic leukemia, chronic lymphatic leukemia, acute leukemias, acute myelotic leukemia, acute lymphatic leukemia and lymphomas.

10. The method of claim 7, wherein the cancer is liver cancer.

11. The method of claim 7, wherein the cancer is stomach cancer.

12. The method of claim 7, wherein the cancer is cervix carcinoma.

13. A method of treating cancer, comprising administering to a patient i) a thymosin peptide, ii) a purified immune RNA or DNA. iii) one or more astragalus polysaccharides, iv) one or more letinous edodes polysaccharides, v) a cell wall component of Mycobacterium grannis, Mycobacterium phlei or Mycobaterium subtilis vi) a polysaccharide nucleic acid fraction of bacillus Calmette-Guerin vii) a transfer factor viii) one or more non steroidal anti inflammatory agents and ix) an enkephalin chosen from methionine enkephalin or leucine enkephalin.

14. The method of claim 13, wherein the thymosin peptide is thymosin alpha 1 (TA1).

15. The method of claim 13, wherein the enkephalin is methionine enkephalin.

16. The method of claim 13, wherein the cell wall component is from Mycobacterium subtilis.

17. The composition of claim 13, wherein the non steroidal anti inflammatory agent is chosen from the group consisting of aceclofenac, acemetacin, amoxiprin, aspirin, azapropazone, benorilate, bromfenac, carprofen, celecoxib, choline magnesium salicylate, diclofenac, diflunisal, etodolac, etoracoxib, faislamine, fenbuten, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinprazone, suprofen, tenoxicam, tiaprofenic acid, and tolmetin.

18. The method of claim 13, wherein the cancer is chosen from the group consisting of malignant tumors, benign tumors, solid tumors, sarcomas, carcinomas, hyperproliferative disorders, carcinoids, Ewing sarcomas, Kaposi sarcomas, brain tumors, tumors originating from the brain and/or the nervous system and/or the meninges, gliomas, glioblastomas, neuroblastomas, stomach cancer, kidney cancer, kidney cell carcinomas, prostate cancer, prostate carcinomas, connective tissue tumors, soft tissue sarcomas, pancreas tumors, liver tumors, head tumors, neck tumors, laryngeal cancer, esophageal cancer, thyroid cancer, osteosarcomas, retinoblastomas, thymoma, testicular cancer, lung cancer, lung adenocarcinoma, small cell lung carcinoma, bronchial carcinomas, breast cancer, intestinal cancer, colorectal tumors, colon carcinomas, rectum carcinomas, gynaecological tumors, ovary tumors/ovarian tumors, uterine cancer, cervical cancer, cervix carcinomas, cancer of body of uterus, corpus carcinomas, endometrial carcinomas, urinary bladder cancer, urogenital tract cancer, bladder cancer, skin cancer, epithelial tumors, squamous epithelial carcinoma, basaliomas, spinaliomas, melanomas, intraocular melanomas, leukemias, monocyte leukemia, chronic leukemias, chronic myelotic leukemia, chronic lymphatic leukemia, acute leukemias, acute myelotic leukemia, acute lymphatic leukemia and lymphomas.

19. The method of claim 18, wherein the cancer is liver cancer.

20. The method of claim 18, wherein the cancer is stomach cancer.

21. The method of claim 18, wherein the cancer is cervix carcinoma.

22. The method of claim 13, wherein the purified immune RNA or DNA is in injection form.

23. The method of claim 13 wherein the cell wall component of Mycobacterium grannis, Mycobacterium phlei or Mycobaterium subtilis is in injection form.

24. The method of claim 23, wherein the cell wall component is from Mycobaterium subtilis.

25. The method of claim 13, wherein a polysaccharide nucleic acid fraction of bacillus Calmette-Guerin is in injection form.

26. The method of claim 13, wherein the enkephalin chosen from methionine enkephalin or leucine enkephalin is in injection form.

27. The method of claim 26, wherein the enkephalin is methionine enkephalin.

28. The method of claim 13, wherein the thymosin peptide, one or more astragalus polysaccharides, one or more letinous edodes polysaccharides and the transfer factor are each in tablet form.
